SEQUENCE LISTING (99 sequences)

SEQ ID NO: 1 - DNA, 6 bp, Artificial Sequence
  Synthetic ribosome binding site
ggagtg

SEQ ID NO: 2 - DNA, 6 bp, Artificial Sequence
  Synthetic Shine-Dalgarno sequence
gattga

SEQ ID NO: 3 - DNA, 18 bp, Artificial Sequence
  I-SceI recognition sequence
tagggataacagggtaat

SEQ ID NO: 4 - DNA, 18 bp, Artificial Sequence
  Mutant I-SceI recognition sequence
tagggataacaaggtaat

SEQ ID NO: 5 - DNA, 7 bp, Artificial Sequence
  BbvCI recognition sequence
cctcagc

SEQ ID NO: 6 - DNA, 6 bp, Artificial Sequence
  EcoRI recognition sequence
gaattc

SEQ ID NO: 7 - DNA, 11 bp, Artificial Sequence
  BglI recognition sequence
  misc_feature (4)..(8): Xaa denotes any nucleotide
  misc_feature (4)..(8): “n” denotes any nucleotide
gccnnnnnggc

SEQ ID NO: 8 - DNA, 6 bp, Artificial Sequence
  Eco57I recognition sequence
ctgaag

SEQ ID NO: 9 - DNA, 12 bp, Artificial Sequence
  BcgI recognition sequence
  misc_feature (4)..(9): “n” denotes any nucleotide
cgannnnnntgc

SEQ ID NO: 10 - DNA, 8 bp, Artificial Sequence
  AscI recognition sequence
ggcgcgcc

SEQ ID NO: 11 - DNA, 8 bp, Artificial Sequence
  AsiSI recognition sequence
gcgatcgc

SEQ ID NO: 12 - DNA, 8 bp, Artificial Sequence
  FseI recognition sequence
ggccggcc

SEQ ID NO: 13 - DNA, 8 bp, Artificial Sequence
  FspAI recognition sequence
rtgcgcay

SEQ ID NO: 14 - DNA, 8 bp, Artificial Sequence
  NotI recognition sequence
gcggccgc

SEQ ID NO: 15 - DNA, 8 bp, Artificial Sequence
  PacI recognition sequence
ttaattaa

SEQ ID NO: 16 - DNA, 7 bp, Artificial Sequence
  PasI recognition sequence
cccwggg

SEQ ID NO: 17 - DNA, 8 bp, Artificial Sequence
  PmeI recognition sequence
gtttaaac

SEQ ID NO: 18 - DNA, 7 bp, Artificial Sequence
  PpuMI recognition sequence
rggwccy

SEQ ID NO: 19 - DNA, 8 bp, Artificial Sequence
  PspXI recognition sequence
vctcgagb

SEQ ID NO: 20 - DNA, 7 bp, Artificial Sequence
  RsrII recognition sequence
cggwccg

SEQ ID NO: 21 - DNA, 7 bp, Artificial Sequence
  SanDI recognition sequence
gggwccc

SEQ ID NO: 22 - DNA, 8 bp, Artificial Sequence
  SbfI recognition sequence
cctgcagg

SEQ ID NO: 23 - DNA, 7 bp, Artificial Sequence
  SexAI recognition sequence
accwggt

SEQ ID NO: 24 - DNA, 8 bp, Artificial Sequence
  SgrAI recognition sequence
crccggyg

SEQ ID NO: 25 - DNA, 8 bp, Artificial Sequence
  SrfI recognition sequence
gcccgggc

SEQ ID NO: 26 - DNA, 8 bp, Artificial Sequence
  Sse232I recognition sequence
gcccggcg

SEQ ID NO: 27 - DNA, 7 bp, Artificial Sequence
  Sse8647I recognition sequence
aggwcct

SEQ ID NO: 28 - DNA, 8 bp, Artificial Sequence
  SwaI recognition sequence
atttaaat

SEQ ID NO: 29 - protein, 9 aa, Artificial Sequence
  Conserved motif of LAGLIDADG family of homing endonucleases
LAGLIDADG

SEQ ID NO: 30 - protein, 7 aa, Artificial Sequence
  Conserved motif of GIY-YIG family of homing endonucleases
  MISC_FEATURE (4)..(4): Xaa denotes 10-11 of amino acid residues, wherein Xaa can be any amino acid residue
GIYXYIG

SEQ ID NO: 31 - DNA, 24 bp, Artificial Sequence
  I-AniI recognition sequence
ttgaggaggtttctctgtaaataa

SEQ ID NO: 32 - DNA, 20 bp, Artificial Sequence
  I-ApeKI recognition sequence
gcaaggctgaaacttaaagg

SEQ ID NO: 33 - DNA, 24 bp, Artificial Sequence
  I-BasI recognition sequence
agtaatgagcctaacgctcagcaa

SEQ ID NO: 34 - DNA, 28 bp, Artificial Sequence
  I-BmoI recognition sequence
gagtaagagcccgtagtaatgacatggc

SEQ ID NO: 35 - DNA, 29 bp, Artificial Sequence
  I-CeuI recognition sequence
cgtaactataacggtcctaaggtagcgaa

SEQ ID NO: 36 - DNA, 30 bp, Artificial Sequence
  I-ChuI recognition sequence
gaaggtttggcacctcgatgtcggctcatc

SEQ ID NO: 37 - DNA, 18 bp, Artificial Sequence
  I-CmoeI recognition sequence
tcgtagcagctcacggtt

SEQ ID NO: 38 - DNA, 23 bp, Artificial Sequence
  CpaI recognition sequence
cgatcctaaggtagcgaaattca

SEQ ID NO: 39 - DNA, 20 bp, Artificial Sequence
  I-CpaII recognition sequence
cccggctaactctgtgccag

SEQ ID NO: 40 - DNA, 30 bp, Artificial Sequence
  I-CreI, I-CvuI, I-MsoI, I-PakI recognition sequence
ctgggttcaaaacgtcgtgagacagtttgg

SEQ ID NO: 41 - DNA, 30 bp, Artificial Sequence
  I-CsmI recognition sequence
gtactagcatggggtcaaatgtctttctgg

SEQ ID NO: 42 - DNA, 30 bp, Artificial Sequence
  I-DmoI recognition sequence
atgccttgccgggtaagttccggcgcgcat

SEQ ID NO: 43 - DNA, 24 bp, Artificial Sequence
  I-DreI recognition sequence
caaaacgtcgtaagttccggcgcg

SEQ ID NO: 44 - DNA, 24 bp, Artificial Sequence
  I-LlaI recognition sequence
cacatccataaccatatcattttt

SEQ ID NO: 45 - DNA, 22 bp, Artificial Sequence
  PI-MgaI recognition sequence
cgtagctgcccagtatgagtca

SEQ ID NO: 46 - DNA, 29 bp, Artificial Sequence
  PI-MtuI recognition sequence
aacgcggtcggcaaccgcacccgggtcac

SEQ ID NO: 47 - DNA, 21 bp, Artificial Sequence
  I-NanI, I-NitI, I-NjaI recognition sequence
aagtctggtgccagcacccgc

SEQ ID NO: 48 - DNA, 22 bp, Artificial Sequence
  PI-PabI recognition sequence
gggggcagccagtggtcccgtt

SEQ ID NO: 49 - DNA, 23 bp, Artificial Sequence
  PI-PabII recognition sequence
acccctgtggagaggagcccctc

SEQ ID NO: 50 - DNA, 30 bp, Artificial Sequence
  PI-PfuI recognition sequence
gaagatgggaggagggaccggactcaactt

SEQ ID NO: 51 - DNA, 29 bp, Artificial Sequence
  PI-PfuII recognition sequence
acgaatccatgtggagaagagcctctata

SEQ ID NO: 52 - DNA, 19 bp, Artificial Sequence
  PI-PkoI recognition sequence
gattttagatccctgtacc

SEQ ID NO: 53 - DNA, 15 bp, Artificial Sequence
  PI-PkoII recognition sequence
cagtactacggttac

SEQ ID NO: 54 - DNA, 19 bp, Artificial Sequence
  I-PogI, I-Tsp061I recognition sequence
cttcagtatgccccgaaac

SEQ ID NO: 55 - DNA, 25 bp, Artificial Sequence
  I-PorI recognition sequence
gcgagcccgtaagggtgtgtacggg

SEQ ID NO: 56 - DNA, 29 bp, Artificial Sequence
  I-PpoI recognition sequence
taactatgactctcttaaggtagccaaat

SEQ ID NO: 57 - DNA, 30 bp, Artificial Sequence
  PI-PspI recognition sequence
tggcaaacagctattatgggtattatgggt

SEQ ID NO: 58 - DNA, 28 bp, Artificial Sequence
  I-ScaI recognition sequence
tgtcacattgaggtgcactagttattac

SEQ ID NO: 59 - DNA, 30 bp, Artificial Sequence
  PI-ScaI recognition sequence
taagtcgggtgcggagaaagaggaaaagag

SEQ ID NO: 60 - DNA, 24 bp, Artificial Sequence
  F-SceI recognition sequence
gatgctgtaggcataggcttggtt

SEQ ID NO: 61 - DNA, 30 bp, Artificial Sequence
  I-SceI recognition sequence
agttacgctagggataacagggtaatatag

SEQ ID NO: 62 - DNA, 30 bp, Artificial Sequence
  PI-SceI recognition sequence
atctatgtcgggtgcggagaaagaggtaat

SEQ ID NO: 63 - DNA, 20 bp, Artificial Sequence
  F-SceII recognition sequence
ctttccgcaacagtaaaatt

SEQ ID NO: 64 - DNA, 29 bp, Artificial Sequence
  I-SceII recognition sequence
ttttgattctttggtcaccctgaagtata

SEQ ID NO: 65 - DNA, 29 bp, Artificial Sequence
  I-SceIII recognition sequence
attggaggttttggtaactatttattacc

SEQ ID NO: 66 - DNA, 29 bp, Artificial Sequence
  I-SceIV recognition sequence
tcttttctcttgattagccctaatctacg

SEQ ID NO: 67 - DNA, 24 bp, Artificial Sequence
  I-SceV recognition sequence
aataattttcttcttagtaatgcc

SEQ ID NO: 68 - DNA, 24 bp, Artificial Sequence
  I-SceVI recognition sequence
gttatttaatgttttagtagttgg

SEQ ID NO: 69 - DNA, 28 bp, Artificial Sequence
  I-SceVII recognition sequence
tgtcacattgaggtgcactagttattac

SEQ ID NO: 70 - DNA, 25 bp, Artificial Sequence
  I-SpomI recognition sequence
gtggttggacggtatatccaccact

SEQ ID NO: 71 - DNA, 19 bp, Artificial Sequence
  I-Ssp6803I recognition sequence
gtcgggctcataacccgaa

SEQ ID NO: 72 - DNA, 24 bp, Artificial Sequence
  F-TevI recognition sequence
gaaacacaagaaatgtttagtaaa

SEQ ID NO: 73 - DNA, 24 bp, Artificial Sequence
  I-TevI recognition sequence
agtggtatcaacgctcagtagatg

SEQ ID NO: 74 - DNA, 29 bp, Artificial Sequence
  F-TevII recognition sequence
tttaatcctcgcttcagatatggcaactg

SEQ ID NO: 75 - DNA, 30 bp, Artificial Sequence
  I-TevII recognition sequence
gcttatgagtatgaagtgaacacgttattc

SEQ ID NO: 76 - DNA, 30 bp, Artificial Sequence
  I-TevIII recognition sequence
tatgtatcttttgcgtgtacctttaacttc

SEQ ID NO: 77 - DNA, 30 bp, Artificial Sequence
  F-TflI recognition sequence
tggcgacgaaaaccgcttggaaagtggctg

SEQ ID NO: 78 - DNA, 30 bp, Artificial Sequence
  F-TflII recognition sequence
acctaccattaacggagtcaaaggccattg

SEQ ID NO: 79 - DNA, 30 bp, Artificial Sequence
  F-TflIV recognition sequence
taggtactggacttaaaattcaggttttgt

SEQ ID NO: 80 - DNA, 26 bp, Artificial Sequence
  PI-TfuI recognition sequence
tagattttaggtcgctatatccttcc

SEQ ID NO: 81 - DNA, 22 bp, Artificial Sequence
  PI-TfuII, PI-ThyI, PI-TliI recognition sequence
  misc_feature (6)..(6): n is a, c, g, or t
  misc_feature (12)..(12): n is a, c, g, or t
taygcngayacngacggyttyt

SEQ ID NO: 82 - DNA, 30 bp, Artificial Sequence
  PI-TliII recognition sequence
aaattgcttgcaaacagctattacggctat

SEQ ID NO: 83 - DNA, 20 bp, Artificial Sequence
  I-TwoI recognition sequence
tcttgcacctacacaatcca

SEQ ID NO: 84 - DNA, 30 bp, Artificial Sequence
  PI-ZbaI recognition sequence
tacgttggttgtggtgaaagaggaaaagag

SEQ ID NO: 85 - DNA, 53 bp, Artificial Sequence
  Synthetic oligonucleotide
catgcagctagcggagtgaaacgatgcagtttaaggtttacacctataaaaga

SEQ ID NO: 86 - DNA, 51 bp, Artificial Sequence
  Synthetic oligonucleotide
agtacggcatgccgtatgtctagattatattccccaaaacatcaggttaat

SEQ ID NO: 87 - DNA, 54 bp, Artificial Sequence
  Synthetic oligonucleotide
ctagcattacgctagggataacagggtaatatcacgctctagacatacggcatg

SEQ ID NO: 88 - DNA, 46 bp, Artificial Sequence
  Synthetic oligonucleotide
ccgtatgtctagagcgtgatattaccctgttatccctagcgtaatg

SEQ ID NO: 89 - DNA, 54 bp, Artificial Sequence; identical to SEQ ID NO: 87
  Synthetic oligonucleotide

SEQ ID NO: 90 - DNA, 46 bp, Artificial Sequence; identical to SEQ ID NO: 88
  Synthetic oligonucleotide

SEQ ID NO: 91 - DNA, 22 bp, Artificial Sequence
  Synthetic oligonucleotide
tacgtacgatttaaataggcct

SEQ ID NO: 92 - DNA, 32 bp, Artificial Sequence
  Synthetic oligonucleotide
gagctcaggcctgactcactatagggagaccg

SEQ ID NO: 93 - DNA, 31 bp, Artificial Sequence
  Synthetic oligonucleotide
ctagctaggccttaagcgacttcattcacct

SEQ ID NO: 94 - DNA, 31 bp, Artificial Sequence
  Synthetic oligonucleotide
tattaaggcctgctccagtggcttctgtttc

SEQ ID NO: 95 - DNA, 31 bp, Artificial Sequence
  Synthetic oligonucleotide
ataattaggcctcttagagccttcaacccag

SEQ ID NO: 96 - DNA, 44 bp, Artificial Sequence
  Synthetic oligonucleotide
atcagtgaattcaggaaactcgagatgaaaaatattaaaaaaaa

SEQ ID NO: 97 - DNA, 32 bp, Artificial Sequence
  Synthetic oligonucleotide
atgccgggtaccttattttaaaaaagtttcgg

SEQ ID NO: 98 - DNA, 21 bp, Artificial Sequence
  Synthetic oligonucleotide
gatgtaagcagcacccaggat

SEQ ID NO: 99 - DNA, 49 bp, Artificial Sequence
  Synthetic oligonucleotide
  misc_feature (13)..(18): n is a, c, g, or t
ttttgggctagcnnnnnnaaacgatgcagtttaaggtttacacctataa

(19) United States
(12) Patent Application Publication
(10) Pub. No.: US 2007/0042404 A1
(43) Pub. Date: Feb. 22, 2007

(54) Compositions and method for detecting endonuclease activity

(76) Inventors: Huimin Zhao, Champaign, IL (US); Zhilei Chen, New York, NY (US)

Correspondence Address: LICATA & TYRRELL P.C., 66 E. MAIN STREET, MARLTON, NJ, 08053, US

(21) Appl. No.: 11/475,446

(22) Filed: Jun. 27, 2006

Related U.S. Application Data
(60) Provisional application No. 60/709,923, filed on Aug. 19, 2005.

(51) Int. Cl. C12Q 1/68; C07H 21/04; C12P 21/06; C12N 9/22; C12N 15/74; C12N 1/21

(52) U.S. Cl. 435/6; 435/69.1; 435/199; 435/252.3; 435/471; 536/23.2

(57) ABSTRACT

The present invention relates to compositions and a cell-based assay for detecting endonuclease activity. The assay involves the basic principle of linking a endonuclease cleavage event with cell survival. When an endonuclease cleaves its cognate endonuclease recognition site located on a vector containing a toxic reporter protein, the vector is degraded and the cell survives because the toxic reporter protein cannot be produced. A cell employed in the instant assay advantageously expresses a transporter protein that facilitates transport of a regulatory molecule used to induce expression of the toxic reporter protein. Kits comprising the compositions of the instant invention are also provided.

### INTRODUCTION

This application claims the benefit of U.S. Provisional Application No. 60/073,403, filed Feb. 2, 1998, which is herein incorporated by reference in its entirety. This invention was made in the course of research sponsored by the National Science Foundation, Grant No. BES-0348107. The U.S. government may have certain rights in this invention.

### BACKGROUND OF THE INVENTION

Homing endonuclease genes are mobile DNA elements that are made up of introns and inteins (Belfort & Roberts (1997) Nucleic Acids Res. 25:3379-3388). These enzymes recognize specific 14-40 bp DNA sequences and catalyze site-specific double-strand breaks in DNA. Homing endonucleases have great potential to be applied in gene targeting, as the site-specific DNA double-strand breaks that they introduce significantly stimulate homologous recombination (Szostak, et al. (1983) Cell 33:25-35). Gene correction at the mutated locus by means of homologous recombination has clear advantages over virus-driven random integration, which suffers the consequences of transgene silencing and improper activity. However, frequency of homologous recombination events is generally low. Attempts have been made to increase the efficiency of homologous recombination in mammalian cells, e.g., by increasing the size of the DNA sharing homology with the target locus, using isogenic genomic DNA, or improving selection procedures (Jasin (1996) Trends Genet. 12:224-228). As a DNA double-strand break is lethal to cell survival, it triggers cell repair machinery and greatly increases the frequency of homologous recombination at the site of the double-strand break. In recombinant DNA technologies, the use of a homing endonuclease results in enhanced homologous replacement of the gene being targeted, even with relatively short stretches of homologous DNA (Jasin (1996) Trends Genet. 12:224-228; Cohen-Tannoudji, et al. (1998) Mol. Cell Biol. 18:1444-1448).

The limited natural repertoire of target sequences of homing endonucleases hampers the application of these enzymes, and the lack of an efficient selection methods restricts the use of directed evolution approaches for engineering of homing endonucleases with novel sequence specificity (Gimble, et al. (2003) J. Mol. Biol. 334:993-1008; Samuelson & Xu (2002) J. Mol. Biol. 319:673-683). One in vivo selection system for detecting homing endonuclease activity links the catalytic activity of a homing endonuclease to the survival of E. coli via a DNA cleavage event (Gruen, et al. (2002) Nucleic Acids Res. 30:e29). This system employs two plasmids, one plasmid encodes a mutant barnase gene with two amber (TAG) stop codons under an inducible arabinose promoter, followed by tandem endonuclease recognition sites. The other plasmid contains nucleic acids encoding a homing endonuclease fused to an Amber suppressor tRNA supE under the constitutive lac promoter. The co-expression of the mutant barnase gene and the tRNA expression cassette fusion protein results in cell death. However, the cleavage of the target DNA sequence by the homing endonuclease, before arabinose-mediated induction of mutant barnase expression, can eliminate the plasmid encoding mutant barnase, resulting in cell survival. While this system can be applied to assay homing endonuclease activity in vivo, the drawbacks of this system include a relatively high background survival (survival of cells harboring both plasmids in the presence of arabinose and absence of homing endonuclease activity) and a low sensitivity (cells with the wild-type homing endonuclease I-SceI exhibit a cell survival rate of 25% in the presence of two tandem copies of the original I-SceI recognition site). These disadvantages impede the use of this system in directed evolution of homing endonucleases with modified specificity toward recognition sequences.

Needed in the art is a sensitive cell based assay for detecting endonuclease activity. The present invention meets this long-felt need.

### SUMMARY OF THE INVENTION

The present invention is a recombinant host cell containing an inducible promoter operably linked to a nucleic acid molecule encoding an endonuclease, and a vector containing at least one selected endonuclease recognition site and a small molecule-regulated promoter operably linked to a nucleic acid encoding a toxic reporter protein. In one embodiment, the recombinant host cell recombinantly expresses a transporter protein which transports the small molecule.

The present invention is also a vector system for detecting endonuclease activity. The vector system contains a vector harboring at least one selected endonuclease recognition site and a small molecule-regulated promoter operably linked to a nucleic acid encoding a toxic reporter protein. In one embodiment, the vector system further contains a vector harboring an inducible promoter operably linked to a nucleic acid molecule encoding an endonuclease. In another embodiment, the vector system further harbors nucleic acids encoding a transporter protein. Kits encompassing the vector system of the invention are also provided.

The present invention is further a method for detecting the activity of an endonuclease. The method involves inducing in a recombinant host cell of the instant invention the expression of the endonuclease, contacting the host cell with a small molecule so that toxic reporter protein expression is induced, and determining whether the host cell grows in the presence of the small molecule thereby detecting the activity of the endonuclease.

### DETAILED DESCRIPTION OF THE INVENTION

A sensitive, low background, cell-based assay for detecting endonuclease activity has now been developed. The assay involves the basic principle of linking a double-stranded DNA cleavage event with cell survival. The instant cell-based assay employs a vector, referred to herein as a reporter vector or reporter plasmid, harboring at least one selected endonuclease recognition site and nucleic acids encoding a toxic reporter protein, the expression of which is under the control of a small molecule-regulated promoter. On another vector, or optionally chromosomally integrated, is a nucleic acid encoding an endonuclease, the expression of which is under the control of an inducible promoter. A cell employed in the instant assay can additionally express a recombinant transporter protein, which facilitates transport of the regulatory small molecule. In particular embodiments, the promoter controlling expression of the transporter protein is not regulated by the small molecule.

The instant assay is carried out by inducing expression of the endonuclease in the host cell, wherein if the endonuclease recognizes the selected endonuclease recognition site, a double-strand break occurs in the reporter vector. This double-strand break results in degradation of the reporter vector so that toxic reporter protein expression is blocked and the cell survives. Conversely, when the endonuclease fails to recognize the selected endonuclease recognition site of the reporter vector, the vector is not degraded and the toxic reporter protein is expressed in the presence of the regulatory small molecule. Toxic reporter protein expression results in cell death. Advantageously, when the host cells expresses a small molecule transporter protein whose expression is independent of the presence of the small molecule, the small molecule is efficiently transported into the cell to provide tight regulation of the toxic reporter protein. Transport protein expression results in a decrease in background cell growth attributed to cells that survive in the absence of plasmid degradation and presence of small molecule.

By way of illustration, the instant assay was used to assay the activity of homing endonuclease I-SceI. The toxic reporter protein employed was CcdB. CcdB was identified as a 101 amino acid gene product of the F-plasmid (Bahassi, et al. (1995) Mol. Microbiol. 15:1031-1037). CcdB poisons DNA gyrase and is responsible for the killing of F-plasmid-free segregants during cell division (Bahassi, et al. (1995) supra; Loris, et al. (1999) J. Mol. Biol. 285:1667-1677). Reporter plasmid p11-ccdB-wtx1 was employed. This plasmid encoded the toxic reporter protein CcdB under control of the arabinose-inducible BAD promoter, followed by one copy of the I-SceI endonuclease recognition site. A second plasmid, pTrc-ISceI, contained nucleic acids encoding homing endonuclease I-SceI under control of the Trc promoter so that I-SceI expression was inducible by IPTG. The cleavage of the reporter plasmid by I-SceI linearized the reporter plasmid and caused it to be quickly degraded inside the host E. coli cell by RecA (Kuzminov & Stahl (1997) J. Bacteriol. 179:880-888). Thus, the expression of CcdB was eliminated before induction of I-SceI expression by arabinose, and cells survived.

One feature of the instant assay is the tight regulation of CcdB expression so that it only exerts its toxic effect upon arabinose induction. While, the BAD promoter was selected because of its high induction ratio and tight regulation by arabinose (Guzman, et al. (1995) J. Bacteriol. 177:4121-4130), one of skill in the art will appreciate that other tightly regulated inducible promoters can also be employed to control CcdB expression. CcdB was cloned into pBAD18 with the ribosome binding site 5′-GGAGTG-3′ (SEQ ID NO:1) obtained from pBAD18s (Guzman, et al. (1995) supra) to generate pBAD-ccdB. The pBAD18 plasmid contains the pBR322 origin of replication and maintains 100-300 copies per cell. Transformation of pBAD-ccdB into E. coli BW25141 resulted in much slower cell growth in Luria-Bertani (LB) medium containing 100 μg/mL ampicillin and low cell survival rates on agar plates, indicating that the low level of CcdB expression even under BAD promoter was still toxic to E. coli cells. To further decrease intracellular CcdB concentration, the level of ccdB translation was decreased by modifying the ribosome binding site. By reducing the ribosome binding site strength, mutant p11-ccdB, which displayed normal cell growth on LB+100 μg/mL ampicillin plates and no cell growth on LB+100 μg/mL ampicillin+4 mM arabinose, was selected. DNA sequence analysis of this mutant revealed its Shine-Dalgarno sequence to be 5′-GATTGA-3′ (SEQ ID NO:2). One copy of the original I-SceI recognition sequence was then inserted after ccdB in p11-ccdB to form the reporter plasmid p11-ccdB-wtx1.

The second plasmid, pTrc-ISceI, encoding homing endonuclease I-SceI under the inducible transcriptional control of the Trc promoter, had a p15a origin of replication and was maintained in E. coli at ˜15 copies per cell. I-SceI was used to demonstrate the linkage between DNA cleavage and cell survival. Host cells harboring the reporter plasmid (p11-ccdB-wtx1) were transformed with either the plasmid containing homing endonuclease (pTrc-ISceI) or a control plasmid (pTrc-p15a) and subsequently assayed for endonuclease activity (Table 1). Transformation of host cells with pTrc-ISceI resulted in a survival rate of 80-100%, whereas transformation with pTrc-p15a plasmid resulted in a survival rate of only 0.3-0.9%. As an additional control, the active site residue Asp44 was mutated to alanine to generate an inactive I-SceI variant (Asp44Ala; Moure, et al. (2003) J. Mol. Biol. 334:685-695). Strains harboring a plasmid encoding the inactive I-SceI variant (pTrc-D44A) had a survival rate of 0.5-0.9%, similar to that of pTrc-p15a. To demonstrate endonuclease specificity, the recognition site of I-SceI was modified from 5′-TAG GGA TAAˆCAG GGT AAT-3′ (SEQ ID NO:3) to 5′-TAG GGA TAA CAa GGT AAT-3′ (SEQ ID NO:4), wherein the lowercase “a” designates the single base mutation which disrupts recognition and cleavage by I-SceI (Monteilhet, et al. (1990) Nucleic Acids Res. 18:1407-1413). Transformation of host cells with a plasmid encoding this mutant recognition site (p11-mISceI) in combination with pTrc-ISceI resulted in a survival rate of less than 0.02%. These results indicate that the in vivo assay of the instant invention can used to efficiently link a DNA cleavage event to cell survival through DNA sequence specificity and the activity of an endonuclease such as I-SceI.

TABLE 1

| | Survival Rate¹ | | |
|---|---|---|---|
| Host Cell | pTrc-IsceI | pTrc-p15a | pTrc-D44A |
| p11-wtx1 | 80-100% | 0.3-0.9% | 0.5-0.9% |
| p11-LacY-wtx1 | 80-100% | <0.003% | ND² |
| p11-mISceI | <0.02% | ND | ND |

¹Cells transformed with the plasmids indicated were plated on LB + 50 μg/mL kanamycin, with or without 10 mM arabinose. Survival rate was calculated by dividing the number of colonies formed on arabinose-containing plates by the number of colonies formed on the kanamycin only plate, after accounting for dilution factors.
²ND, not determined.

As the survival rate of cells harboring only one copy of the I-SceI recognition site was very high (80-100%), introduction of two tandem copies of the I-SceI recognition site to the reporter plasmid did not yield significantly higher survival rates. However, it is contemplated that depending on the endonuclease, the level of endonuclease expression, and host cell being assayed, the sensitivity of the instant in vivo assay can be increased by increasing the concentration of the endonuclease recognition site.

Transformation of host cells harboring p11-ccdB-wtx1 with control plasmid (pTrc-p15a) resulted in a ˜0.3-0.9% cell survival rate and longer recovery at 37° C. shaker after electroporation, resulting in even higher background survival rates. Although such background survival can be greatly reduced by shortening the post-transformation recovery time in liquid SOC medium at 37° C., transformation efficiency and the survival rate of the host cells transformed with pTrc-ISceI also decreases. This background survival is likely due to the partial or complete loss of the reporter plasmid in the absence of antibiotic selection pressure (ampicillin) during recovery in liquid SOC medium and on selection plates containing only kanamycin and arabinose.

The BAD promoter is known to be subject to all-or-none induction, because genes encoding the arabinose transporters (araE and araFGH) are also under the regulation of the BAD promoter (Smolke, et al. (2001) Appl. Microbiol. Biotechnol. 57:689-696). This autocatalytic mechanism causes only a fraction of the cells in the population to be fully induced while the remaining cells stay uninduced for an extended time period (Siegele & Hu (1997) Proc. Natl. Acad. Sci. USA 94:8168-8172). This mechanism may also delay the overall induction of gene expression from the BAD promoter. Accordingly, a mutant LacY gene (Ala177Cys) was introduced into the host cells as an additional arabinose transporter (Morgan-Kiss, et al. (2002) Proc. Natl. Acad. Sci. USA 99:7373-7377). LacY(Ala177Cys) was placed on the reporter plasmid under regulatory control of the lac promoter, resulting in plasmid p11-LacY-wtx1. Host cells harboring this plasmid were transformed with pTrc-p15a or pTrc-ISceI and endonuclease activity was assayed. The survival rate of cells harboring p11-LacY-wtx1 and pTrc-p15a was less than 0.003% (Table 1), 100- to 300-fold lower than that of host cells containing p11-ccdB-wtx1. Further, a survival rate of 80-100% was obtained for cells harboring p11-LacY-wtx1 and pTrc-ISceI.

To evaluate the efficiency of this system for identifying active homing endonuclease variants, host cells harboring p11-LacY-wtx1 were transformed with a plasmid mixture containing a 1:104 molar ratio of pTrc-ISceI:pTrc-D44A and subject to the assay disclosed herein. A total of 1.2×10⁶ clones were screened and 60 colonies were observed on LB+50 μg/mL kanamycin+10 mM arabinose plates (i.e., selection plates). Four random clones were selected and DNA sequence analysis conducted. Two of the selected clones contained pTrc-ISceI, indicating a 5000-fold enrichment. All colonies formed on the selection plates were pooled together and grown in LB+50 μg/mL kanamycin media overnight. Their plasmids were isolated, re-transformed into the same host cell strain and an aliquot was plated on LB+50 μg/mL kanamycin+10 mM arabinose. Four random clones from the selection plate were subject to DNA sequence analysis and all four clones contained pTrc-ISceI. These data indicate that extremely rare functional clones from a library of clones can be readily identified using the instant in vivo assay, and therefore this assay is suitable for detecting mutant endonucleases generated by directed evolution.

The instant assay is a significant improvement over existing methods for detecting endonuclease activity. The instant assay is simple in that only one protein, i.e., the toxic reporter protein, is required to confer the toxic phenotype. Furthermore, the instant assay does not require the expression of the target endonuclease as an N-terminal fusion protein. Moreover, the instant assay is highly sensitive. For example, using the instant assay, the survival rate of cells expressing wild-type I-SceI and one copy of the original I-SceI endonuclease recognition site was 80-100%. Further, by introducing in the host cells a small molecule transporter protein, whose expression is independent of the presence of the small molecule, the background cell survival rate is less than 0.003%. In view of these advantages, the instant assay can be used for analyzing recognition site specificity of a known or newly identified endonuclease, or in high throughput assays for detecting endonucleases with modified specificity of endonuclease recognition sites generated by directed evolution of the endonuclease.

Accordingly, the instant invention provides compositions and a cell-based method for detecting endonuclease activity. As used in the context of the instant invention, an endonuclease is an enzyme that specifically recognizes or binds to its nucleic acid substrate (e.g., DNA) at internal sites in the nucleic acid and catalyzes site-specific double-strand breaks. The internal sites are interchangeably referred to herein as endonuclease recognition sites or recognition sequences. In particular embodiments of the instant invention, the double-strand break is near to (e.g., within 1-30 nucleotides) or within the recognition sequence.

Endonucleases, which can be detected in accordance with the instant invention include, but are not limited to, DNA mismatch-specific endonucleases, restriction endonucleases, and homing endonucleases.

In general, DNA mismatch-specific endonucleases are plant-derived endonucleases which nick or cleave duplex DNA at insertion/deletion and base-substitution mismatches (Oleykowski, et al. (1998) Nucl. Acid Res. 26:4597-4602; Yang, et al. (2000) Biochem. 39:3533-3541; Kulinski, et al. (2000) BioTechniques 29:44-48; Colbert, et al. (20001) Plant Physiol. 126:480-484; Sokurenko, et al. (2001) Nucl. Acids Res. 29:e111; U.S. Pat. No. 5,869,245). In particular embodiments, the DNA mismatch-specific endonuclease being assayed in the instant invention creates a double-strand break at the site of DNA mismatch. Exemplary endonucleases of this type include, but are not limited to, SP endonuclease from spinach (Oleykowski, et al. (1999) Biochemistry 38:2200-2205) and CEL II from celery (Qui, et al. (2004) Biotechniques 36(4):702-7).

Restriction endonucleases are traditionally classified into three types based upon subunit composition, cleavage position, sequence-specificity and cofactor-requirements. Type I restriction endonucleases are complex, multisubunit, combination restriction-and-modification enzymes that cut DNA at random far from their recognition sequences. Type III restriction endonucleases are large combination restriction-and-modification enzymes that cleave outside of their recognition sequences and require two such sequences in opposite orientations within the same DNA molecule to accomplish cleavage of particular interest in the context of the instant invention are type II restriction endonucleases. Type II restriction endonucleases cut DNA at defined positions close to or within their recognition sequences. The most common type II restriction endonucleases include HhaI, HindIII and NotI that cleave DNA within their recognition sequences. Most type II enzymes recognize DNA sequences that are symmetric because they bind to DNA as homodimers, but a few, (e.g., BbvCI, 5′-CCTCAGC-3′; SEQ ID NO:5) recognize asymmetric DNA sequences because they bind as heterodimers. Some enzymes recognize continuous, uninterrupted sequences (e.g., EcoRI, 5′-GAATTC-3′; SEQ ID NO:6) in which the two half-sites of the recognition sequence are adjacent, while others recognize discontinuous sequences (e.g., BglI, 5′-GCCNNNNNGGC-3′; SEQ ID NO:7) in which the half-sites are separated. Cleavage leaves a 3′-hydroxyl on one side of each cut and a 5′-phosphate on the other. They tend to be small enzymes, with subunits in the 200-350 amino acid range.

The next most common type II restriction endonucleases, usually referred to as “type IIs”, include enzymes such as FokI and AlwI that cleave outside of their recognition sequence to one side. These enzymes are intermediate in size, 400-650 amino acids in length, and they recognize sequences that are continuous and asymmetric. They encompass two distinct domains, one for DNA binding and the other for DNA cleavage.

The third major kind of type II restriction endonucleases, also referred to as “type IV”, are large, combination restriction-and-modification enzymes, 850-1250 amino acids in length, in which the two enzymatic activities reside in the same protein chain. These enzymes cleave outside of their recognition sequences. Those that recognize continuous sequences (e.g., Eco57I, 5′-CTGAAG-3′; SEQ ID NO:8) cleave on just one side, whereas those that recognize discontinuous sequences (e.g., BcgI, 5′-CGANNNNNNTGC-3′; SEQ ID NO:9) cleave on both sides releasing a small fragment containing the recognition sequence.

As will be appreciated by one of skill in the art, a restriction endonuclease of the instant invention should not cut the genomic DNA of the genome of the host cell of the assay. Accordingly, type II restriction endonucleases which can readily be detected in accordance with the instant assay contain at least a six or seven nucleotide recognition sequence or more desirably at least an eight to twelve nucleotide recognition sequence. In one embodiment, the recognition sequence of the instant endonuclease is continuous and at least seven, eight, ten, or twelve nucleotides in length. Alternatively stated, the endonuclease is a rare-cutting enzyme. Exemplary restriction endonucleases which can be detected using the instant assay include, but are not limited the endonucleases listed in Table 2.

TABLE 2

| Restriction | Recognition | |
|---|---|---|
| Endonuclease | Sequence (5′-> 3′) | SEQ ID NO: |
| AscI | GG\hat{ }CGCGCC | 10 |
| AsiSI | GCGAT\hat{ }CGC | 11 |
| FseI | GGCCGG\hat{ }CC | 12 |
| FspAI | RTGC\hat{ }GCAY | 13 |
| NotI | GC\hat{ }GGCCGC | 14 |
| PacI | TTAAT\hat{ }TAA | 15 |
| PasI | CC\hat{ }CWGGG | 16 |
| PmeI | GTTT\hat{ }AAAC | 17 |
| PpuMI | RG\hat{ }GWCCY | 18 |
| PspXI | VC\hat{ }TCGAGB | 19 |
| RsrII | CG\hat{ }GWCCG | 20 |
| SanDI | GG\hat{ }GWCCC | 21 |
| SbfI | CCTGCA\hat{ }GG | 22 |
| SexAI | A\hat{ }CCWGGT | 23 |
| SgrAI | CR\hat{ }CCGGYG | 24 |
| SrfI | GCCC\hat{ }GGGC | 25 |
| Sse232I | GC\hat{ }CCGGCG | 26 |
| Sse8647I | AG\hat{ }GWCCT | 27 |
| SwaI | ATTT\hat{ }AAAT | 28 |

\hat{ } indicates cleavage site. R = A or C; Y = C or T; W = A or T; V = A, C, or G; B = C, G, or T.

As exemplified herein, the instant assay is particularly suitable for the detection of homing endonucleases. Accordingly, an embodiment of the instant invention is the use of the disclosed assay to detect the activity of a homing endonuclease. Homing endonucleases are a group of endonucleases encoded by mobile intervening sequences. The mobile intervening sequences (e.g., intron or intein) are laterally transferred to homologous alleles via activity of the homing endonuclease encoded thereby. Homing endonucleases are characterized by their ability to bind long DNA target sites (14-40 bp) and create double-stranded breaks. Homing endonucleases are classified into four families, the LAGLIDADG family, named for containing one or two copies of the conserved Leu-Ala-Gly-Leu-Ile-Asp-Ala-Asp-Gly (SEQ ID NO:29) motif (Belfort & Roberts (1997) Nucl. Acids. Res. 25:3379-3388) and conserved three-dimensional structure; the GIY-YIG family, characterized by the conserved Gly-Ile-Tyr-(Xaa)₁₀₋₁₁-Tyr-Ile-Gly (SEQ ID NO:30) motif (Kowalski, et al. (1999) Nucl. Acids. Res. 27:2115-2125); the His-Cys box family, characterized by a highly conserved series of histidines and cysteines over a central 100 residue region (Johansen, et al. (1993) Nucl. Acids. Res. 21:4405; Muscarella, et al. (1990) Mol. Cell. Biol. 10:3386-3396); and the HNH family which contains two pairs of conserved histidines surrounding a conserved asparagines within a 30-33 residue sequence (Shub, et al. (1994) Trends Biochem. Sci. 19:402-404; Gorbalenya (1994) Protein Sci. 3:1117-1120). Exemplary homing endonucleases and recognition sequences known to be recognized and cleaved by the cognate endonuclease are listed in Table 3.

TABLE 3

| Restriction | | SEQ ID |
|---|---|---|
| Endonuclease | Recognition Sequence (5′->3′) | NO: |
| I-AniI | TTGAGGAGGTTTCTCTGTAAATAA | 31 |
| I-ApeKI | GCAAGGCTGAAACTTAAAGG | 32 |
| I-BasI | AGTAATGAGCCTAACGCTCAGCAA | 33 |
| I-BmoI | GAGTAAGAGCCCGTAGTAATGACATGGC | 34 |
| I-CeuI | CGTAACTATAACGGTCCTAAGGTAGCGAA | 35 |
| I-ChuI | GAAGGTTTGGCACCTCGATGTCCGCTCATC | 36 |
| I-CmoeI | TCGTAGCAGCTCACGGTT | 37 |
| I-CpaI | CGATCCTAAGGTAGCGAAATTCA | 38 |
| I-CpaII | CCCGGCTAACTCTGTGCCAG | 39 |
| I-CreI | CTGGGTTCAAAACGTCGTGAGACAGTTTGG | 40 |
| I-CsmI | GTACTAGCATGGGGTCAAATGTCTTTCTGG | 41 |
| I-CvuI | CTGGGTTCAAAACGTCGTGAGACAGTTTGG | 40 |
| I-DmoI | ATGCCTTGCCCGGTAAGTTCCGGCGCGCAT | 42 |
| H-DreI | CAAAACGTCGTAAGTTCCGGCGCG | 43 |
| I-LlaI | CACATCCATAACCATATCATTTTT | 44 |
| PI-MgaI | CGTAGCTGCCCAGTATGAGTCA | 45 |
| I-MsoI | CTGGGTTCAAAACGTCGTGACACAGTTTGG | 40 |
| PI-MtuI | AACGCGGTCGGCAACCGCACCCGGGTCAC | 46 |
| I-NanI | AAGTCTGGTGCCAGCACCCGC | 47 |
| I-NitI | AAGTCTGGTGCCAGCACCCGC | 47 |
| I-NjaI | AAGTCTGGTGCCAGCACCCGC | 47 |
| PI-PabI | GGGGGCAGCCAGTGGTCCCGTT | 48 |
| PI-PabII | ACCCCTGTGGAGAGGAGCCCCTC | 49 |
| I-PakI | CTGGGTTCAAAACGTCGTGAGACAGTTTGG | 40 |
| PI-PfuI | GAAGATGGGAGGAGGGACCGGACTCAACTT | 50 |
| PI-PfuII | ACGAATCCATGTGGAGAAGAGCCTCTATA | 51 |
| PI-PkoI | GATTTTAGATCCCTGTACC | 52 |
| PI-PkoII | CAGTACTACGGTTAC | 53 |
| I-PogI | CTTCAGTATGCCCCGAAAC | 54 |
| I-PorI | GCGAGCCCGTAAGGGTGTGTACGGG | 55 |
| I-PpoI | TAACTATGACTCTCTTAAGGTAGCCAAAT | 56 |
| PI-PspI | TGGCAAACAGCTATTATGGGTATTATGGGT | 57 |
| I-ScaI | TGTCACATTGAGGTGCACTAGTTATTAC | 58 |
| PT-ScaI | TAAGTCGGGTGCGGAGAAAGAGGAAAAGAG | 59 |
| F-SceI | GATGCTGTAGGCATAGGCTTGGTT | 60 |
| I-SceI | AGTTACGCTAGGGATAACAGGGTAATATAG | 61 |
| PI-SceI | ATCTATGTCGGGTGCGGAGAAAGAGGTAAT | 62 |
| F-SceII | CTTTCCGCAACAGTAAAATT | 63 |
| I-SceII | TTTTGATTCTTTGGTCACCCTGAAGTATA | 64 |
| I-SceIII | ATTGGAGGTTTTGGTAACTATTTATTACC | 65 |
| I-SceIV | TCTTTTCTCTTGATTAGCCCTAATCTACG | 66 |
| I-SceV | AATAATTTTCTTCTTAGTAATGCC | 67 |
| I-SceVI | GTTATTTAATGTTTTAGTAGTTGG | 68 |
| I-SceVII | TGTCACATTGAGGTGCACTAGTTATTAC | 69 |
| I-SpomI | GTGGTTGGACGGTATATCCACCACT | 70 |
| I-Ssp6803I | GTCGGGCTCATAACCCGAA | 71 |
| F-TevI | GAAACACAAGAAATGTTTAGTAAA | 72 |
| I-TevI | AGTGGTATCAACGCTCAGTAGATG | 73 |
| F-TevII | TTTAATCCTCGCTTCAGATATGGCAACTG | 74 |
| I-TevII | GCTTATGAGTATGAAGTGAACACGTTATTC | 75 |
| I-TevIII | TATGTATCTTTTGCGTGTACCTTTAACTTC | 76 |
| F-TflI | TGGCGACGAAAACCGCTTGGAAAGTGGCTG | 77 |
| F-TflII | ACCTACCATTAACGGAGTCAAAGGCCATTG | 78 |
| F-TflIV | TAGGTACTGGACTTAAAATTCAGGTTTTGT | 79 |
| PI-TfuI | TAGATTTTAGGTCGCTATATCCTTCC | 80 |
| PI-TfuII | TAYGCNGAYACNGACGGYTTYT | 81 |
| PI-ThyI | TAYGCNGAYACNGACGGYTTYT | 81 |
| PI-TliI | TAYGCNGAYACNGACGGYTTYT | 81 |
| PI-TliII | AAATTGCTTGCAAACAGCTATTACGGCTAT | 82 |
| I-Tsp061I | CTTCAGTATGCCCCGAAAC | 54 |
| I-TwoI | TCTTGCACCTACACAATCCA | 83 |
| PI-ZbaI | TACGTTGGTTGTGGTGAAAGAGGAAAAGAG | 84 |

N = A, C, T or G.

Desirably, the endonuclease of the instant assay only cleaves the selected recognition site intentionally inserted in the reporter vector and does not cleave the host cell genome or any other nucleotides of the assay vector(s). A host cell genome or vector containing a recognition site of an endonuclease of interest can be mutated or genetically engineered using convention methods to destroy the unwanted recognition site so that the only recognition site capable of being cleaved by the endonuclease is that intentionally inserted in the reporter vector. Alternatively, a host cell or vector can be selected which does not contain the recognition site cleaved by the endonuclease of interest. To illustrate, detection of SwaI can be carried out in a Pseudomonas aeruginosa host cell (Wozniak, et al. (1995) Appl. Environ. Microbiol. 61:1739-44; Reetz & Jaeger (1998) Chem. Phys. Lipids 93:3-14) which does not contain a recognition site for SwaI in its genomic sequence (Stover, et al. (2000) Nature 406:959-964), whereas detection of I-SceI can be carried out in E. coli as exemplified herein. Nucleic acid sequences of vectors (e.g., obtained from commercial sources or GENBANK) and host cell genomes (e.g., obtained from the NCBI Genome database) can be readily analyzed by the skilled artisan to determine the number and location of selected endonuclease recognition sequences in the vector or host cell genome.

Nucleic acid molecules encoding the above listed endonucleases are well-known to the skilled artisan. An endonuclease nucleic acid molecule can be obtained by conventional cloning techniques using restriction digests of cloned genes or by employing the polymerase chain reaction (PCR), using appropriate primers which result in amplification of the endonuclease coding region. Sources of endonuclease cloned genes and sequences thereof can be readily obtained from the GENBANK and REBASE databases of the world-wide web. For example, nucleic acids encoding the I-SceI endonuclease encoded by a mobile group I intron within the mitochondrial COX1 gene of Saccharomyces cerevisiae is available under GeneID No. 854596. Likewise, the nucleic sequence encoding, e.g., PakI, SgrAI, AsiSI, I-PpoI, and I-TevII are available under GENBANK Accession Nos. L44125, AF290880, AY261807, M38131, and Y00122, respectively.

A nucleic acid molecule encoding the endonuclease of the instant assay is positioned adjacent to and under the control of an inducible promoter. Wherein the endonuclease is a heterodimer, nucleic acids encoding both subunits can be co-expressed from the same or different inducible promoter. For example, an internal ribosome binding site can be introduced between the open reading frames of nucleic acids encoding two subunits of a heterodimer such that one inducible promoter is used and the resulting polycistronic message is translated into the two individual subunits. It is understood in the art that to bring the coding sequence under the control of such a promoter, the transcriptional unit of the endonuclease is operably linked or positioned between about 1 and 50 nucleotides downstream of (i.e., 3′ of) the selected promoter. Accordingly, a promoter is said to be operably linked to a nucleic acid molecule when the promoter mediates transcriptional regulation of the nucleic acid molecule into mRNA for subsequent translation into protein.

An inducible promoter is one in which the rate of RNA polymerase binding and initiation is modulated by external stimuli. The inducible promoter can be of genomic origin or synthetically generated and a variety of inducible promoters suitable for use in the instant assay are well-known in the art. For example, inducible promoters for use in prokaryotic and eukaryotic host cells include, but are not limited to, those responsive to heavy metal ions such as copper (Shetty, et al. (2004) Biotechnol. Bioeng. 88(5):664-70), heat shock (Nouer, et al. (1991) In: Heat Shock Response, ed. Nouer, CRC, Boca Raton, Fla., pp. 167-220), hormones (Lee, et al. (1981) Nature 294:228-232; Hynes, et al. (1981) Proc. Natl. Acad. Sci. USA 78:2038-2042; Klock, et al. (1987) Nature 329:734-736; Israel and Kaufman (1989) Nucl. Acids Res. 17:2589-2604; No, et al. (1996) Proc. Natl. Acad. Sci. USA 93:3346-3351), light (U.S. Pat. No. 6,733,996) or agents such as tetracycline (Gatz, et al. (1992) Plant J. 2:397-404; U.S. Pat. No. 5,814,618) or IPTG (Labow, et al. (1990) Mol. Cell. Biol. 10:3343-3356; Baim, et al. (1991) Proc. Natl. Acad. Sci. USA 88:5072-5076). Any inducible promoter can be selected for controlling expression of the endonuclease so long as the external stimulus is not the same as the small molecule used to induce expression of the toxic reporter protein; endonuclease expression is prior to, and independent of, toxic reporter protein expression. An exemplary inducible promoter for use in a prokaryotic host cell such as E. coli is the Trc promoter which is responsive to IPTG.

Depending on the application of the instant method, the endonuclease nucleic acid molecule can be chromosomally or extrachromosomally located, e.g., on a vector such as a plasmid, BAC, or shuttle vector. For example, a host cell recombinantly modified to contain a chromosomally located endonuclease nucleic acid molecule can be readily transformed with a vector harboring a selected endonuclease recognition site and a nucleic acid encoding a toxic reporter protein, and used to analyze the length and sequence requirements for sequence recognition of a particular endonuclease. Alternatively, if the endonuclease is to be subjected to random mutation or directed evolution, it may be desirable to have the endonuclease nucleic acid molecule located on a vector for ease of manipulation (e.g., mutation and sequence analysis). When the endonuclease nucleic acid molecule is located on a vector, the choice of vector may depend on such factors as the host cell to be transformed, the vector copy number per cell desired, and/or the level of gene expression desired. A suitable vector for expressing an endonuclease of the instant assay can include a vector derived from a pBR322 vector, a YCp vector, a pET vector, a pUC vector, or a p15a vector, e.g., as exemplified herein.

The instant assay also employs a vector containing at least one selected endonuclease recognition site and a small molecule-regulated promoter operably linked to a nucleic acid encoding a toxic reporter protein, i.e., the reporter vector. A selected endonuclease recognition site is one which is selected for being specific for a particular endonuclease or is selected for being a target sequence for which an endonuclease is desired (e.g., generation of an endonuclease by directed evolution). Exemplary selected endonuclease recognition sites for particular endonucleases are listed in Tables 2 and 3. The location of the selected endonuclease recognition site within the vector is not critical nor is the number of copies of the recognition site. In particular embodiments, the reporter vector contains at least one selected endonuclease recognition site. In other embodiments, the reporter vector contains two, three, four, five, or more selected endonuclease recognition sites.

To achieve high rates of survival of the host cell, desirably no expression of the toxic reporter protein is detectable in the absence of the small molecule. Accordingly, the small molecule-regulated promoter should be tightly regulated. Tight regulation can be achieved using several strategies including, selecting an appropriate small molecule-regulated promoter or, as exemplified herein, decreasing the strength of the ribosome binding site, or a combination thereof. Small-molecule regulated promoters which can be employed include the heavy metal ions, hormones, and small molecules (i.e., tetracycline and IPTG) disclosed above, as well as those responsive to sugars such arabinose (Khlebnikov, et al. (2000) J. Bacteriol. 182(24):7029-7034). Desirably, the small-molecule regulated promoter is responsive to an external stimulus different than that of the inducible promoter controlling expression of the endonuclease. An exemplary small-molecule regulated promoter for use in a prokaryotic host cell such E. coli is the BAD promoter. The BAD promoter has rapid kinetics (Johnson & Schleif (1995) J. Bacteriol. 177:3438-3442), an induction ratio of up to 1,200-fold (Guzman, et al. (1995) supra) and tight control (Haldimann, et al. (1998) J. Bacteriol. 180:1277-1286).

Ribosome binding site engineering was identified herein as a simple and effective way to modify protein expression levels, as a wide range of protein expression levels was observed in the library screen. This mechanism has been widely used by nature when an operon is regulated by a single promoter and the members of the operon are expressed at various levels according to their own Shine-Dalgarno sequence.

The reporter vector can be derived from any vector with the proviso that it is capable of being degraded by the host cell upon introduction of a double-strand break by the endonuclease. As with the endonuclease-encoded vector, the choice of reporter vector can be dependent upon the host cell being transformed, the vector copy number per cell desired, and/or the level of gene expression desired. A suitable reporter vector for expressing a toxic reporter protein of the instant assay can include a vector derived from a pUC8 vector, a YEp vector, or a pBR322 vector, e.g., as exemplified herein.

A toxic reporter protein is any protein, which either by itself or in the presence of a substrate, results in cell death upon its expression. Examples of toxic reporter proteins which can be employed in the instant assay, include, but are not limited to, control of cell death B (ccdB), cytosine deaminase in the presence of 5-fluorocytosine, tetracycline resistance gene (Tet^{r}) in the presence of NiCl₂ (Podolsky, et al. (1996) Plasmid 36:112-115), and the Bacillus subtilis levansucrase-encoding gene (sacB) in the presence of sucrose (Gay, et al. (1985) J. Bacteriol. 164:918-921). Nucleic acids encoding these toxic reporter proteins are well-known in the art and, as with the other nucleic acids of the instant invention, can be readily obtained using conventional cloning or PCR methodologies in accordance with standard practices. See, e.g., Current Protocols in Molecular Biology, Ausubel et al. 1995. 4th edition, John Wiley and Sons; PCR Strategies, M. A. Innis, D. H. Gelf, and J. J. Sninsky (eds.), 1995. Academic Press.

To decrease the background survival of cells in the absence of plasmid degradation and presence of the small molecule, the instant assay can, optionally, be carried out with host cells which express a transporter protein which facilitates uptake of the small molecule required for regulating expression of the toxic reporter protein. In particular embodiments, the promoter operably linked to nucleic acids encoding the transporter protein is not regulated by the small molecule, i.e., expression of the transporter protein is constitutive or, alternatively, induced in the presence of a stimulus that is not the small molecule. The nucleic acid molecule encoding the transporter protein can be chromosomally or extrachromosomally located, e.g., located on the reporter vector harboring the toxic reporter protein. Suitable transporter proteins can be selected by one of skill in the art based upon the small molecule being employed to induce toxic reporter protein expression. For example, a copper transport protein can be employed when the small molecule regulator is copper (Puig, et al. (2002) J. Biol. Chem. 277:26021-26030). By way of illustration, a mutant LacY(Ala177Cys) (Morgan-Kiss, et al. (2002) supra) under regulatory control of the lac promoter was found to significantly reduce background survival rate in the presence of the small molecule regulator, arabinose. Advantageously, the transporter protein facilitates the homogeneous induction of the toxic reporter protein by the small molecule within the cell population, forces the immediate induction by the small molecule upon plating on the small molecule-containing plates, and raises the intracellular small molecule concentration thereby increasing transcription efficiency of each individual small molecule-regulated promoter so that cells with decreased reporter plasmid copy number can still produce enough toxic reporter protein to kill the cell.

As one of skill in the art will appreciate, prokaryotic host cells such as Escherichia coli, Pseudomonads such as Pseudomonas aeruginosa (Wozniak, et al. (1995) supra), and Lactococcus lactis (Nouaille, et al. (2003) Genet. Mol. Res. 2(1):102-111) are desirably employed in the instant assay because of their small genome size, which reduces the likelihood of there being a selected endonuclease recognition site located therein. However, it is contemplated that in certain applications a eukaryotic host cell with a relatively small genome can also be readily used. For example, fungal host cells including yeasts such as Pichia pastoris, Saccharomyces cerevisiae, Schizosaccharomyces pombe, Pichia pastoris, Kluveromyces lactis, Hansenula Polymorpha, and Candida albicans (Fleer (1992) Curr. Opin. Biotech. 3(5):486-496) and filamentous fungi such as Neurospora crassa, Aspergillus nidulins, and Aspergillus fumigatus, can be used in the detection of homing endonucleases. Accordingly, in particular embodiments of the instant invention, a microbial host cell is employed. As used herein, a microbial host cell refers to a bacterial or fungal (e.g., yeast) cell. In other embodiments, a bacterial host cell is used in the instant assay.

Chromosomal or extrachromosomal introduction of the nucleic acid molecules and vectors disclosed herein into the appropriate host cell can be carried out using conventional methods of transformation, calcium phosphate co-precipitation, DEAE-dextran-mediated transfection, lipofection, microinjection, polyethylene glycol-mediated transformation, Agrobacterium-mediated transformation, cell fusion, and ballistic bombardment. Suitable methods for transforming host cells may be found in Sambrook, et al. (Molecular Cloning: A Laboratory Manual, 2nd Edition, Cold Spring Harbor Laboratory press (1989)) and other laboratory manuals. Selection and identification of recombinant host cells containing the nucleic acid molecules and vectors can be carried out using conventional selectable markers (e.g., drug resistance markers on the vectors) in combination with nucleic acid detection techniques (e.g., PCR amplification or Southern blot analysis).

Having developed a vector system (i.e., a reporter vector harboring an endonuclease recognition site and nucleic acids encoding a toxic reporter protein, and a vector harboring nucleic acids encoding an endonuclease) and a recombinant host cell containing the same, the present invention is also a method of using the recombinant host cell in a method for detecting the activity of an endonuclease. The method of the invention involves inducing in the recombinant host cell the expression of the endonuclease, contacting the host cell with a small molecule which induces expression of a toxic reporter protein from the reporter vector, and determining whether the host cell can grow in the presence of the small molecule. If the endonuclease cleaves the reporter vector at the endonuclease recognition sequence, the reporter vector will be destroyed (i.e., nucleic acids encoding the toxic reporter protein will be degraded) and the host will survive in the presence of the small molecule. If the endonuclease fails to recognize and cleave the endonuclease recognition sequence, the reporter vector remains intact allowing expression of the toxic reporter protein in the presence of the small molecule and the host will not grow in culture. The instant method is useful for characterizing endonuclease recognition sites of known and newly identified endonucleases as well as analyzing mutant endonucleases. Advantageously, when the host cell of the instant method expresses a small molecule transporter protein, background cell survival is very low in the presence of the small molecule providing a very highly sensitive method for detecting endonucleases which have been modified by directed evolution. Thus, in one embodiment of the instant method, the nucleic acids encoding the endonuclease have been subjected to mutagenesis. See, e.g., Seligman, et al. (2002) Nucl. Acids Res. 30:3870-9. Endonucleases modified and detected in accordance with the instant method can be used in gene targeting (e.g. homologous recombination), gene therapy, DNA cloning and transgenic approaches in mammalian and plant systems.

The present invention is also a kit for use in accordance with the instant method. A kit of the invention includes one or more of the vectors of the invention. Desirably, the kit includes a vector, i.e., reporter vector, containing at least one selected endonuclease recognition site and a small molecule-regulated promoter operably linked to a nucleic acid encoding a toxic reporter protein. In one embodiment, the kit further contains a vector harboring an inducible promoter operably linked to a nucleic acid molecule encoding an endonuclease. Alternatively, the kit contains cells harboring the reporter vector and/or the vector encoding the nuclease, or optionally a chromosomal copy of the endonuclease nucleic acids. In another embodiment, the reporter vector of the kit further harbors nucleic acids encoding a transporter protein, wherein transcription of the nucleic acids encoding the transporter protein is not regulated by the small molecule. The cells/vectors of the kit are provided in a suitable formulation or as a lyophilized powder, and the kits can also contain, or be packaged with, one or more further molecular biological reagents (e.g., sterile water), and instructions for use.

The invention is described in greater detail by the following non-limiting examples.

### EXAMPLE 1

### Vector Construction

To construct plasmid pBAD-ccdB, ccdB gene was amplified from pTCKJ02 (Carrier, et al. (1998) Biotechnol. Bioeng. 59:666-672) with primers NheI-ccdB (5′-cat gca gct agc GGA GTG aaa cga tgc agt tt aag gtt tac acc tat aaa aga-3′, SEQ ID NO:85; NheI site is underlined) and ccdB-R(X+S) (5′-agt acg gca tgc cgt atg tct aga tta tat tcc cca aaa cat cag gtt aat, SEQ ID NO:86; XbaI and SphI sites are underlined). The resulting amplicon was cloned into the NheI and SphI sites of pBAD18 (Guzman, et al. (1995) supra). Primer NheI-ccdB also contained the Shine-Dalgarno sequence from pBAD18s (Guzman, et al. (1995) supra), designated by capital letters. For ribosome binding site engineering, the Shine-Dalgarno sequence was replaced by six N's to form a random library and the mutant with the desired activity was designated p11-ccdB. The recognition sequence of I-SceI was inserted into p11-ccdB at XbaI and SphI sites using oligonucleotides 5′-cta gca tta cgc TAG GGA TAA CAG GGT AAT atc acg ctc tag aca tac ggc atg-3′ (SEQ ID NO:87) and 5′-ccg tat gtc tag agc gtg atA TTA CCC TGT TAT CCC Tag cgt aat g-3′ (SEQ ID NO:88), wherein the recognition sequence of I-SceI is shown in capital letters and XbaI and SphI sites are underlined. The resulting vector was designated p11-ccdB-wtx1. A modified I-SceI recognition sequence was similarly inserted into p11-ccdB using oligonucleotides 5′-cta gca tta cgc TAG GGA Taa CAG GGT AAT atc acg ctc tag aca tac ggc atg-3′ (SEQ ID NO:89) and 5′-ccg tat gtc tag agc gtg atA TTA CCC TGt TAT CCC Tag cgt aat g-3′ (SEQ ID NO:90), wherein the modified recognition sequence of I-SceI is shown in capital letters and XbaI and SphI sites are underlined. The resulting vector was designated p11-mISceI. Ligation of these oligonucleotides into p11-ccdB XbaI and SphI sites abolished the original XbaI site and introduced a new XbaI site (underlined). Accordingly, additional endonuclease recognition sites can be inserted into the new XbaI and SphI sites.

A synthetic cassette, 5′-tac gta cga ttt aaa tag gcc t-3′ (SEQ ID NO:91), was ligated into the NdeI-ClaI fragment of p11-ccdB-wtx1 to introduce a StuI site (underlined). Mutant LacY(A177C) under transcriptional regulation of the lac promoter was amplified from pLacYA177C (Morgan-Kiss, et al. (2002) supra) with primers plac-StuI (5′-gag ctc agg cct gac tca cta tag gga gac cg-3′, SEQ ID NO:92) and LacY-StuI-R (5′-cta gct agg cct taa gcg act tca ttc acc t-3′, SEQ ID NO:93; StuI site is underlined) and inserted into the StuI site of p11-ccdB-wtx1 to generate p11-LacY-wtx1.

Plasmid pTrc99a was used as a template for cloning nucleic acids encoding I-SceI. The ampicillin resistance gene in pTrc99a was replaced by kanamycin resistance gene to form pTrc-KM. The large fragment of pTrc99a was digested with BspHI and treated with T4 DNA polymerase (NEW ENGLAND BIOLABS®, Beverly, Mass.) to form blunt ends. Subsequently, this fragment was ligated with the blunt-ended small fragment of pET26b, digested with AlwNI and XhoI. The pBR322 origin of replication in pTrc-KM was replaced by the p15a origin to form pTrc-p15a. The large fragment of pTrc-KM, generated by AlwNI and HpaI digestion, was treated with T4 DNA polymerase and ligated with the StuI-digested PCR product of pACYCDuet-1 (NOVAGEN®, Madison, Wis.) amplified with primers StuI-D1 (5′-tat taa ggc ctg ctc cag tgg ctt ctg ttt c-3′, SEQ ID NO:94; StuI site is underlined) and StuI-D2 (5′-ata att agg cct ctt aga gcc ttc aac cca g-3′, SEQ ID NO:95; StuI site is underlined). Homing endonuclease I-SceI was amplified from pSCM525 (Perrin, et al. (1993) EMBO J. 12:2939-2947) with primers EcoRI-SceI′ (5′-atc agt gaa ttc agg aaa ctc gag atg aaa aat att aaa aaa aa-3′, SEQ ID NO:96; EcoRI site is underlined) and KpnI-Isce-2-C (5′-atg ccg ggt acc tta ttt taa aaa agt ttc gg-3′, SEQ ID NO:97; KpnI site is underlined), and cloned into the KpnI and EcoRI sites of pTrc-p15a to generate pTrc-ISceI. pTrc-D44A, containing a mutant I-SceI (Asp44Ala) was constructed using a megaprimer PCR technique (Sarkar & Sommer (1990) Biotechniques 8:404-407). A first PCR was performed with primers D44A′-C (5′-gat gta agc agc acc cag gat-3′; SEQ ID NO:98) and EcoRI-SceI′ using pSCM525 as template to form a megaprimer. This megaprimer, in combination with KpnI-Isce-2-C, was used to amplify pSCM525 to obtain mutant I-SceI (Asp44Ala), which was subsequently cloned into pTrc-p15a.

### EXAMPLE 2

### Ribosome Binding Site Strength Engineering

CcdB gene was amplified from pTCKJ02 with primers rbs-ccdB-F (5′-ttt tgg gct agc nnn nnn aaa cga tgc agt tta agg ttt aca cct ata a-3′, SEQ ID NO:99; NheI site is underlined) and ccdB-R(X+S), and cloned into pBAD-ccdB digested with NheI and SphI. The −6 to −12 bases of ccdB gene were randomized with degenerate primers to form the random library of Shine-Dalgarno sequences. This library was transformed into BW25141 (lacI^{q} rrnB_{T14} ΔlacZ_{WJ16} ΔphoBR580 hsdR514 ΔaraBAD_{AH33} ΔrhaBAD_{LD78} galU95 endA_{BT333} uidA(ΔMluI)::pir⁺ recA1; Datsenko & Wanner (2000) Proc. Natl. Acad. Sci. USA 97:6640-6645; Lessard, et al. (1998) Chem. Biol. 5:489-504) and plated on LB plates in the presence of 100 μg/mL ampicillin. Colonies formed on the plates were transferred with sterile toothpicks into round-bottom 96-well plates (Evergreen Scientific, Los Angeles, Calif.) containing 50 μL LB+100 μg/mL ampicillin and grown overnight at 37° C. with vigorous shaking. The overnight culture was then diluted with 200 μL per well sterile, double-distilled water. Five μL of this culture was transferred to the corresponding wells of two sterile flat-bottom 96-well plates (RAININ INSTRUMENT®, Oakland, Calif.) containing 200 μL of LB+100 μg/mL ampicillin with or without 10 mM arabinose and incubated at 37° C. for 16 hours. OD₆₀₀ readings were taken using a SPECTRAMAX® 340PC plate reader (Molecular Devices, Sunnyvale, Calif.). Due to the toxic nature of ccdB, it was contemplated that a strong ribosome binding site would lead to little or no cell growth in the absence of arabinose induction, while a weak ribosome binding site would allow high cell survival rates even at the maximum arabinose concentration. A total of 800 colonies were picked and 13 clones showing healthy growth in LB medium without arabinose and little growth in the presence of 10 mM arabinose were selected. These clones were then plated on LB plates containing 100 μg/mL ampicillin and 0 mM, 1 mM, 4 mM or 10 mM arabinose, and incubated at 37° C. for 24 hours. Clone p11-ccdB, which showed normal colony growth on the plate with 0 mM arabinose and no colony on plates with 4 mM arabinose, was chosen for subsequent analyses. DNA sequence analysis of this clone revealed its Shine-Dalgarno sequence as 5′-GATTGA-3′ (SEQ ID NO:2).

### EXAMPLE 3

### In vivo Activity Assay

Host cells were prepared by transforming E. coli strain BW25141 with the appropriate reporter plasmid and plating on a LB+100 μg/mL ampicillin plate. A single colony from the plate was selected and grown in 500 mL of LB+100 μg/mL ampicillin. Electrocompetent cells of these transformed host cells were prepared following standard protocols (Dower, et al. (1988) Nucleic Acids Res. 16:6127-6145). Cells were not grown to log phase at any stage during competent cell production. Typically, 50 μL of the competent, transformed host cells were subsequently transformed with 1-100 ng of pTrc-ISceI/D44A or pTrc-p15a plasmids and the cultures were immediately recovered in SOC media and shaken at 37° C. for 5 minutes. The culture was then diluted 5-fold with warm SOC media. For host cells lacking nucleic acids encoding LacY(Ala177Cys), cells were induced with 0.1 mM isopropyl-β-D-thiogalactopyranoside (IPTG) (Sigma, St. Louis, Mo.) and shaken at 37° C. for 40 minutes before plating on agar plates. For the host cells containing p11-LacY-wtx1, a final concentration of 0.5 mM IPTG was used to induce protein expression from Trc and lac promoters. These cells were allowed to grow at 37° C. for 70 minutes followed by a one hour incubation at 30° C. prior to plating on agar plates. An aliquot of cells was placed on plates of LB+50 μg/mL kanamycin to estimate the total number of transformants. A second aliquot was plated on LB+50 μg/mL kanamycin+10 mM arabinose plates. All plates were incubated at 37° C. for 12-24 hours until colonies were clearly visible. Colonies were counted manually to estimate the survival rate. The survival rate was calculated by dividing the number of colonies formed on the arabinose-containing plate by the number of colonies on the kanamycin only plate, after accounting for dilution factors.

What is claimed is:

1. A recombinant host cell comprising
an inducible promoter operably linked to a nucleic acid molecule encoding an endonuclease, and
a vector containing at least one selected endonuclease recognition site and a small molecule-regulated promoter operably linked to a nucleic acid encoding a toxic reporter protein.

2. The recombinant host cell of claim 1, wherein the host cell recombinantly expresses a transporter protein which transports the small molecule.

3. A vector system for detecting endonuclease activity comprising a vector comprising at least one selected endonuclease recognition site and a small molecule-regulated promoter operably linked to a nucleic acid encoding a toxic reporter protein.

4. The vector system of claim 3, further comprising a vector comprising an inducible promoter operably linked to a nucleic acid molecule encoding an endonuclease.

5. The vector system of claim 3, wherein the vector further comprises nucleic acids encoding a transporter protein which transports the small molecule.

6. A method for detecting the activity of an endonuclease comprising inducing in the recombinant host cell of claim 1 the expression of the endonuclease, contacting the host cell with the small molecule so that toxic reporter protein expression is induced, and determining whether the host cell grows in the presence of the small molecule thereby detecting the activity of the endonuclease.

7. The method of claim 6, wherein nucleic acids encoding the endonuclease have been subjected to directed evolution.

8. A kit comprising a vector comprising at least one selected endonuclease recognition site and a small molecule-regulated promoter operably linked to a nucleic acid encoding a toxic reporter protein.

9. The kit of claim 8, further comprising a vector comprising an inducible promoter operably linked to a nucleic acid molecule encoding an endonuclease.

10. The kit of claim 8, wherein the vector further comprises nucleic acids encoding a transporter protein which transports the small molecule.
